(19) United States
(12) Patent Application Publication
(10) Pub. No.: US 2026/0085099 A1
(43) Pub. Date: Mar. 26, 2026

(54) Isolation of Osteopontin and Glycomacropeptide from Whey

(71) Applicants: UNIVERZA V LJUBLJANI, Ljubljana (SI); ARHEL Projektiranje in Inzeniring D.O.O., Komenda (SI)

(72) Inventors: Jernej Oberckal, Ljubljana (SI); Bojana Bogovic Matijasic, Ljubljana (SI); Maja Zupancic Justin, Komenda (SI)

(21) Appl. No.: 19/111,893

(22) PCT Filed: Sep. 15, 2023
(86) PCT No.: PCT/EP2023/075384

§ 371 Date: Mar. 14, 2025

(30) Foreign Application Priority Data
Sep. 16, 2022 (LU) LU502808

(51) Int. Cl. C07K 14/47

(52) U.S. Cl. CPC C07K 14/4702; C07K 14/4732

(57) ABSTRACT

The present invention generally relates to the dairy industry, and specifically to the isolation and purification of osteopontin and glycomacropeptide, wherein is provided with a process for the isolation of osteopontin (OPN) or glycomacropeptide (GMP) from whey or a whey derived liquid comprising the steps of absorption onto a monolithic chromatography column, an optional washing of the column and subsequent elution of said OPN or GMP using a change in salt concentration and/or pH.

### TECHNICAL FIELD OF THE INVENTION

The present invention relates to the dairy industry, and specifically to milk proteins such as osteopontin and glycomacropeptide and processes for their isolation from whey and similar milk-based matrices.

### BACKGROUND OF THE INVENTION

Whey, the liquid by-product of the dairy industry, accounts for up to 90% of wastewater from dairy farms, and its production amounts to 200 million tonnes per year worldwide, much of which is treated as waste. Due to its high organic matter content, it presents a problem when released into the environment. Whey is especially a problem for small dairies because they cannot afford the expensive technology required to process whey.

There are several varieties of whey produced by different technologies of milk coagulation. The two most common types are sweet and acid whey. Acid whey is produced in the manufacturing of fresh curd cheese or Greek-type yoghurt. The lowering of pH caused by the addition of acids such as HCl, citric acid, lactic acid or others, or by fermentation with lactic acid bacteria, solubilises some of the calcium phosphates that cross-links the casein micelles. This reduces the electrostatic repulsion of micelles, and due to hydrophobic interactions, protein aggregates are formed that make up the curd.

Sweet whey is produced during cheese manufacture by adding the proteolytic enzyme chymosin to the milk to aggregate the caseins. Chymosin breaks down the protein kappa-casein. The resulting insoluble portion of the kappa-casein drives the coagulation of the milk. During coagulation, the cheese curd is formed, and sweet whey is released by syneresis. The soluble phosphorylated and glycosylated portion of kappa-casein, glycomacropeptide (GMP), is released into the sweet whey. GMP is therefore found mainly in sweet whey and in lower concentrations in milk and acid whey. Proteolytic cleavage of kappa-casein does not affect the pH of milk, so sweet whey usually retains the original pH of the milk. Another source of GMP may be a proteolytic digest of kappa-casein. GMP has various biological activities, such as antimicrobial and anticancer activity. It is used in food supplements and functional foods. Because it lacks the amino acid phenylalanine, GMP is suitable for the diet of people with phenylketonuria, who cannot metabolise phenylalanine.

Osteopontin (OPN) is found in all types of whey, but the main source for its industrial extraction is acid whey. OPN is phosphorylated and glycosylated. It is found in various tissues, including muscle and bone, as well as in milk. It has multiple calcium-binding sites and acts as a promotor of bone growth. It also has many other functions. It is most commonly used as an additive in infant formula. Research shows that OPN in infant formula stimulates the development of the baby's immune system and shortens the duration of fever. Due to its versatile activities, OPN is also useful for adults.

Different variants of OPN (truncated, cleaved) have different activities. Some research suggests that cleaved OPN has higher activity in some applications.

For the purification of individual whey proteins, adsorption methods such as high-performance liquid chromatography (HPLC) are often used. These methods rely on the partitioning of solutes into two phases. The solution (mobile phase) is passed through the chromatographic column containing the stationary phase. This often consists of porous particles derivatized with functional groups that represent binding sites for the molecules of interest. The stationary phase may also be in the form of a porous monolith through which the solution is passed. By interacting with the binding sites, the molecules can adsorb to the stationary phase and become separated from the bulk of the solution and can also be concentrated. In a complex solution such as whey, which contains many different molecules, there are molecules with different affinities for the binding sites. When the solution is passed over the stationary phase, competition for binding to the binding sites occurs, and stronger binding molecules displace weaker binding molecules. If the column is overloaded, it will become saturated with mostly strongly binding substances, even if they are present in low concentrations in the incoming solution. If the molecules of interest are of high molecular weight, such as proteins, the displacement of weakly bound proteins by strongly binding proteins is more pronounced when using monolithic columns (and loading them to their capacity (or beyond)) than particle-based columns. The binding sites of the latter are mostly located in dead-end pores, into which the substances enter by diffusion. Since proteins can diffuse less efficiently into the pores at high flow rates, these binding sites are not saturated, and displacement is less efficient than with monolithic columns, where convective flow occurs in the channels where the binding sites are located. Therefore, displacement on a monolithic column can be used to purify the strongly binding proteins to a greater extent than when using particle-based columns.

Bayless (1997) discloses the isolation and biological properties of osteopontin from bovine milk using DEAE-Sephacel beads (weak anion exchanger).

Azuma et al. (2006) purified osteopontin from bovine milk using DEAE-Sephacel and subsequent HPLC with a POROS HQ/M column.

WO2012/117119 discloses a process where osteopontin is isolated by anion exchange in high yield and high purity from complex milk-derived feeds despite the presence of competing proteins in the feed.

WO2002028194 discloses a process for producing a GMP isolate and a β-lactoglobulin enriched WPI from a feedstock containing whey proteins, including GMP and β-lactoglobulin.

The common problems in isolating proteins from complex organic matrices are, for example, expensive and complicated extraction procedures, inactivation of the product or destruction of its structure during extraction, large amounts of waste material from the extraction, expensive equipment, and unusable starting material after the extraction.

In the specific context of isolating OPN and GMP from whey and whey-derived liquids, it is challenging that a substantial proportion of whey is produced at rather small dairies where technically complicated and expensive equipment for the isolation of OPN and GMP is not feasible, and the storage and transportation of whey to central plants for the isolation of OPN and GMP are also not economically feasible.

Hence, there is a need for simple industrial processes for isolating OPN and GMP from whey and whey-derived liquids.

### SUMMARY OF THE INVENTION

The object of the present invention is to provide relatively simple processes for isolating OPN or GMP from whey or whey-derived liquids, which are more efficient than the present processes and which can be used even in medium to small dairies. It is also an object to provide such processes for isolation of both OPN and GMP. More particularly, it is an object of the present invention to provide optimised processes for the isolation of the two proteins from whey, such as to provide high-quality and high economic value products used in functional foods, food supplements, medicines and other purposes. By isolating the proteins from the whey, which is a by-product of dairies, whey can better be valorised.

It is further an object of the present invention to provide processes for isolation of OPN from whey or a whey-derived liquid which has a GMP concentration which is higher than the concentration of OPN.

It is also an object of the present invention to provide such processes for isolating OPN and GMP, which use buffers that pose minimal risk to the environment and can be reused for purification, thus reducing the environmental impact of producing the purified proteins.

The present invention thus provides in a first aspect a process for the isolation of osteopontin (OPN) or glycomacropeptide (GMP) from whey or a whey-derived liquid comprising the steps of absorption onto a monolithic chromatography column, an optional washing of the column and subsequent elution of said OPN or GMP using a change in salt concentration and/or pH.

The present invention may be summarised by the following items:
1. A process for the isolation of osteopontin (OPN) or glycomacropeptide (GMP) from whey or a whey-derived liquid comprising the steps of absorption onto a monolithic chromatography column, an optional washing of the column and subsequent elution of said OPN or GMP using a change in salt concentration and/or pH.
2. The process according to item 1, wherein the monolithic column is loaded with whey or whey-derived liquid with such flow rate and in such quantity that OPN displaces more weakly binding substances from the column.
3. The process according to any one of items 1-2, wherein the monolithic column is loaded with whey or whey-derived liquid with a flow rate in the range from about 0.1 mL per mL of column volume per minute to about 20 mL per mL of column volume per minute, or in the range from about 0.1 mL per mL of column volume per minute to about 5 mL per mL of column volume per minute.
4. The process according to any one of items 1-3, wherein the process is conducted including the washing of the column.
5. The process according to any of items 1-4, wherein said elution comprises the use of an eluent having a higher conductivity than said whey or whey derived liquid loaded onto said monolithic chromatography column.
6. The process according to item 5, wherein said eluent has a conductivity which is at least the conductivity of whey, at least 125% of the conductivity of whey or at least 150% of the conductivity of whey.
7. The process according to any of items 5-6, wherein said eluent has a conductivity in the range from about the conductivity of said whey or whey derived liquid to about 150% of the ionic strength of said whey or whey derived liquid, from about 125% of the conductivity of said whey or whey derived liquid to about 200% of the conductivity of said whey or whey-derived liquid, or has a conductivity which is at least 200% of the conductivity of said whey or whey-derived liquid.
8. The process according to any of items 1-7, wherein said elution comprises the use of an eluent having a pH lower than the pH of said whey or whey-derived liquid loaded onto said monolithic chromatography column.
9. The process according to item 8, wherein said elution comprises the use of an eluent having a pH which is less than about 5.0, less than about 4.5, or less than about 4.0.
10. The process according to any of items 8-9, wherein said elution comprises the use of an eluent having a pH in the range from about 3.0 to about 5.5, or in the range from about 3.5 to about 5.0.
11. The process according to any of items 1-10, wherein said elution is performed by the use of a change in salt concentration and a change in pH.
12. The process according to item 11, wherein the elution is performed by a step change of the salt concentration and a gradient change of the pH.
13. The process according to item 11, wherein the elution is performed by a step change of the pH and a gradient change of the salt concentration.
14. The process according to any of items 1-13, wherein OPN and GMP are eluted separately such that separate fractions are obtained where one fraction comprises the majority of said OPN and a second fraction comprises the majority of said GMP.
15. The process according to any of the items 1-14, wherein said absorption onto a monolithic chromatography column is a high-performance liquid chromatography (HPLC), such as anion-exchange HPLC.
16. The process according to any of items 1-15, wherein said whey or whey derived liquid is filtered prior to said absorption, such as by microfiltration.
17. The process according to any of items 1-16, wherein the flow rate of said whey or whey-derived liquid during the adsorption onto said monolithic chromatography column is sufficiently high to bring the concentration of OPN in the flow-through to a concentration in the range from about 2% to 20% of the corresponding concentration in said whey or whey derived liquid, or in the range from about 2% to 10% of the corresponding concentration in said whey or whey derived liquid.
18, The process according to any of items 1-17, wherein the flow rate during adsorption onto said monolithic chromatography column is at least 5 mL per L column, at least 8 mL per L column or at least 11 mL per L column.
19. The process according to any of items 1-18, wherein the flow-through fraction obtained from the absorption onto said monolithic chromatography column is loaded onto a new monolithic chromatography column or onto the same column for a second process of adsorption of the remaining OPN or GMP.
20. The process according to any of items 1-19, wherein said monolithic chromatography column following the elution is regenerated by a solution having a concentration of sodium chloride in the range from about 0.8M to about 2.0M, and optionally pH in the range from about 2.5 to about 3.5.
21. The process according to any of items 1-20, which is for the isolation of OPN.
22. The process according to any of items 1-20, which is for the isolation of GMP.
23. The process according to any of items 1-22, which is for the isolation of OPN and GMP.
24. A method for preparing OPN or GMP comprising the process as defined in any of items 1-23.
25. The method according to item 24, further comprising a desalination step.
26. The method according to any of items 24-25, comprising a further chromatography step.
27. The method according to any of items 24-26, further comprising a lyophilisation, spray drying or other form of drying to obtain a solid OPN or GMP product.

### DETAILED DESCRIPTION OF THE INVENTION

Unless specifically defined herein, all technical and scientific terms used have the same meaning as commonly understood by a skilled artisan in the fields of dairy sciences, chemistry, food technology and protein process technology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail. Further, the materials, methods, and examples are illustrative only and are not intended to be limiting unless otherwise specified.

The practice of the present invention will employ, unless otherwise indicated, conventional techniques of dairy engineering and protein process technology, which are within the skill of the art. Such techniques are explained fully in the literature. See, for example, the cited references herein.

In an embodiment the process of the present invention uses HPLC for the purification of OPN and/or GMP. The whey, which may vary by type and origin, may optionally be filtered to remove biological matter such as bacteria, which remain after fermentation of the milk, or insoluble protein aggregates. Microfiltration is usually performed at laboratory level (<1 L) with syringe filters with average pore diameters of 0.45 μm or 0.20 μm or tangential flow membrane modules with 750 kDa cut-off or at semi-industrial level (1500 L) with ceramic membranes with pore diameters of 0.5-0.8 μm. The permeate typically contains all soluble proteins (including OPN and GMP) and other substances that are in the incoming whey.

Microfiltered whey may then optionally be concentrated using tangential ultrafiltration with membranes with 10 kDa or 50 kDa cut-off.

In the present embodiment an appropriate amount of microfiltered (and/or concentrated) whey is then fractionated by anion-exchange HPLC using monolithic columns CIMmultus™ (Sartorius—Bia Separations). Due to the use of monolithic carriers and convection, high flow rates can be achieved—up to 20 mL per mL column per minute. Prior to loading of whey, the column is typically equilibrated with at least 10 column volumes of buffer (such as 25 mM sodium acetate) with a pH corresponding to the pH of the whey (such as pH 4.7 for acid whey or pH 5.5 to 6.7 for sweet whey). The whey is then loaded, and through ionic interactions, the proteins GMP and OPN (in the case of whey derived from chymosin-treated milk) or mainly OPN (in the case of acid whey) are adsorbed onto the column and separated from the unbound substances, which flow out of the column as the flow-through fraction. In sweet whey, the GMP concentration is much higher compared to OPN, and the binding sites are soon saturated by GMP (and a small fraction of some other proteins, such as beta-lactoglobulin (B-LG)). GMP (and the other proteins), however, binds weakly to the anion exchanger and is soon displaced by OPN, which binds more strongly. As the column saturation is approached, increasingly more OPN and less GMP are bound. By regulating the amount of sweet whey loaded, it is possible to regulate the amount of bound (and consequently isolated) GMP and OPN. The concentration of GMP in the flow-through fraction is often similar to the loaded whey. The concentration of OPN in the flow-through fraction, however, may be up to 15% of the concentration of OPN in the loaded whey. This remaining OPN can be isolated by reloading the flow-through fraction on a clean column and repeating the same elution process hereby ensuring a very high yield of OPN. This remaining OPN is typically somewhat different from the OPN that binds first to the column: SDS-PAGE analysis shows a relatively greater abundance of the band at 70 kDa, which presumably corresponds to the full-length glycosylated OPN. This full-length OPN, therefore, binds more weakly to the monolithic anion-exchanger.

The composition of the flow-through fraction is typically, apart from substances that bind to the anion exchanger, the same as that of the loaded whey.

In an embodiment the column with the bound proteins may then optionally be washed with a buffer (such as sodium acetate or sodium citrate, pH 5.0) to remove unbound and weakly bound substances. In this step, a very pure fraction (>90% according to SDS-PAGE) of B-LG is eluted when loading sweet whey. The column may then be washed with a buffer (such as sodium acetate or sodium citrate, pH 5.0), which contains a small concentration (such as 100 mM) of sodium chloride. This elutes the GMP (in the case of loading sweet whey) and some other bound proteins. Then the column may be washed with a higher concentration of sodium chloride (such as 400 mM), which elutes OPN. The final washing step of this embodiment is with a high concentration of sodium chloride (such as 0.8M-2M) and, if needed, an acidic pH (such as pH 3) to remove the strongly bound substances. The column is then equilibrated in the initial buffer and ready for the next loading. The column may be periodically washed with a solution of 1M NaOH and 2M NaCl (CIP solution) or 30% isopropanol to remove aggregated or hydrophobic substances and potential microorganisms.

The eluted fractions may then optionally be concentrated and desalted (and/or additionally separated) with ultrafiltration (or diafiltration) with membranes with a suitable pore size, such as 10 kDa cut-off or 50 kDa cut-off. To achieve higher purity, eluted proteins can also be re-chromatographed using the same anion-exchange column or a different column, for example, using a cation-exchanger in a negative chromatography run).

The final desalted fractions may then be spray-dried, lyophilised or dried in another way to produce a solid product.

The main advantage of the present invention is the use of monolithic chromatography columns, which exhibit the following advantages over conventional particle-based columns:
All of the flow in the monolithic column goes through the pores, which means that all of the mass transfer happens by convection, whereas the pores in particle-based columns are a dead end, and substances migrate in by diffusion. Larger molecules, such as proteins, are less able to diffuse into the pores efficiently. Therefore, the dynamic binding capacity for convective flow in the monolithic columns is less dependent on the flow rate than in the particle-based columns.
To ensure higher flow rates, the particle-based columns at the industrial scale are often larger than they have to be regarding their binding capacity for the target substance and are not loaded to their final capacity. Our invention uses loading of whey over the column capacity, which enables the competition of strongly binding substances (such as OPN) for the limited number of binding sites on the column with the weakly binding but more abundant substances (such as GMP, β-LG, α-LA and some other whey proteins that bind to the column). Because of this competition, the weakly bound substances are displaced from the column and this, in the end, produces a product of higher purity.
Monolithic columns allow higher flow rates at lower pressure, enabling faster separations.
Monolithic columns have longer lifespans than particle-based columns.

Besides the advantages offered by the use of monolithic columns for the isolation of OPN or GMP, the process of the present invention also has the following advantages over known processes for isolation of OPN or GMP:
The process of the present invention is able to produce separate fractions of OPN and GMP with high purity, which some of the known processes are not able to do (such as EP2681230B1 and EP2681231A1).
Unlike, for example, WO02/28413A1, the process of the present invention for OPN isolation enables the use of feed that contains a high concentration of GMP. The described process makes use of the presumably higher affinity of OPN for binding to the column compared to GMP. In the process of the present invention, when the column is saturated so that almost all binding sites are filled, OPN will displace GMP from the column despite a much higher concentration of GMP.
As a feed, the process of the present invention can use the whey that is a side product in dairies, including smaller dairies.
The process of the present invention for isolation of OPN or GMP is compatible with processes of isolation of other proteins from milk, colostrum or whey that generate a GMP and/or OPN containing feed with a suitable pH (>4.6) and ionic strength (at or at most slightly above the ionic strength of whey), such as the process of extraction of lactoferrin from whey as disclosed in WO2020094731A1.
In the process of the present invention it is not necessary to add any substance to the whey or change it in any other way prior to isolation of GMP and OPN (unlike in the processes described, for example, in WO01/149741A2, WO2002028194A1, EP0393850A2, U.S. Pat. Nos. 7,259,243B2, 5,280,107A in CN102746393A). However, in the process of the present invention the whey may be subject to e.g. microfiltration prior to application to the monolithic chromatography column.
In the process of the present invention the flow-through fraction, remaining after the extraction of proteins, is unaltered apart from the extraction of the proteins and is, as such applicable for other means. In some of the known processes (such as WO01/149741A2 and CN102746393A), the flow-through fraction contains a higher concentration of some substances and is, therefore, less suitable for further processing and presents an additional burden to the environment.
Unlike the process described in EP2681230B1, the process of the present invention is very specific for isolating OPN in the presence of other proteins at high pH (>5) without the need for adjusting the conductivity of the feed.
As a feed, the process of the present invention can use sweet or acid whey or whey concentrate without the need for pH adjustment to a certain value (such as described in WO01/149741A2 or U.S. Pat. No. 5,280,107A).
The process of the present invention uses environmentally-friendly chemicals (such as sodium acetate, sodium citrate, sodium chloride and water), unlike some of the processes known in the art, for example WO2009027284A1, which uses sodium phosphate buffers. The buffers can be used multiple times. The solutions with the high salt content can be used for other purposes (for example, as drive solutions for osmosis).

### Certain Other Definitions

The term “whey” as used herein is intended to mean a liquid which is released from milk after coagulation of milk proteins by acid treatment, by microbial fermentation or by enzymatic treatment with chymosin or other enzyme.

The term “acid whey” as used herein is intended to mean a liquid which is released from milk by acid treatment or by microbial fermentation with lactic acid bacteria.

The term “sweet whey” as used herein is intended to mean a liquid which is released from milk by enzymatic treatment with chymosin or other enzyme.

The term “whey-derived liquid” as used herein is intended to mean a liquid wherein the majority is derived from whey. Non-limiting examples of a whey-derived liquid are concentrated whey, diluted whey, the product obtained by removing certain proteins, e.g. lactoferrin, from whey and the like.

The term “monolithic chromatography column” as used herein is intended to mean a chromatographic column containing a solid phase which is coherent and has many channels inside the solid phase. This is in contrast to chromatographic columns having particulate material as the stationary phase. Monolith materials may be inorganic or organic materials. Non-limiting examples of monolithic chromatography columns are silica-based monoliths and polymer-based monoliths.

### EXAMPLES

### Analytical Methods

Fractions eluted during the anion exchange chromatography were analysed using RP-HPLC, SDS-PAGE (using a specific dye for sialylated and phosphorylated proteins as well as silver nitrate) and mass spectrometry.

The first characterisation step is already the preparative anion exchange chromatography. The exchanger separates substances according to their surface charge. It binds substances with a negative charge. Those with a higher negative charge density are bound more strongly. This effect is able to distinguish between OPN and GMP. GMP elutes at a lower salt concentration than OPN. OPN has several variants, the largest of which (seen on an SDS-PAGE gel at 70 kDa) elutes earlier than the other variants in a linear gradient, but it cannot be completely separated from them.

### RP-HPLC

Several RP-HPLC methods were developed to distinguish between OPN, GMP and other common whey and milk proteins: beta-lactoglobulin (B-LG), alpha-lactalbumin (A-LA), bovine serum albumin (BSA), immunoglobulin G (IgG), alpha-S-casein (ASCN), beta-casein (BCN), kappa-casein (KCN), lactoferrin (LF) and lactoperoxidase (LPO).

The commercially obtained proteins mentioned above and all isolated fractions were analysed using the Phenomenex C8 Widepore Aeris (3.6 μm, 200 Å, 250×4.6 mm) column. Mobile phase A consisted of 0.1% TFA in dH₂O or 0.1% TFA and 5% acetonitrile (ACN) in dH₂O. Mobile phase B consisted of 0.1% TFA in ACN or 0.1% TFA in 90% ACN in dH₂O.

Protein samples were mixed with mobile phase A in a 1:1 ratio or prepared as described in Bobe et al. (1998), who added guanidinium HCl, dithiothreitol, and citrate for better resolution. The methods used were standard RP-HPLC methods with a linear gradient over an appropriate range for the determination of OPN, GMP and the other proteins of interest.

In all cases (with both versions of sample preparation), OPN and GMP were clearly separated in the chromatograms from all other analysed proteins (OPN and GMP always eluted before all other analysed commercial proteins). However, they were not clearly separated from each other. Both OPN and GMP had multiple peaks (both, isolated and commercial proteins), likely due to different forms and degrees of glycosylation and phosphorylation (since both the isolated as well as commercial OPN and GMP were obtained from a pooled milk source and thus from cows that were genetically different from each other), but perhaps also due to proteolysis. GMP and OPN, however, were distinguishable from each other on the basis of the shape of their respective peaks in the chromatogram.

Since OPN and GMP could not be satisfactorily separated by RP-HPLC, a multidimensional approach to characterisation was used. RP-HPLC allows the calculation of the ratio of absorbance at 280 nm and 214 nm. GMP has almost non-existent absorbance at 280 nm due to the lack of aromatic amino acids, while OPN absorbs well at this wavelength. In addition to the peak shape, this allowed further differentiation between the two proteins.

### SDS-PAGE

According to the literature, GMP occurs most frequently as a band at 21 kDa, but also at 14 kDa and other molecular weights up to 45 kDa (Sharma et al., 2021). According to the literature, OPN is present in three bands on an SDS-PAGE gel, at 70, 45 and 35 kDa (Azuma et al., 2005, Bissonnette et al., 2012, Christensen et al., 2020).

We used 12.5% polyacrylamide gels and the TRIS-glycine buffer system (Laemmli, 1970) with standard protocols. We successfully stained both proteins with Coomassie brilliant blue R250. We also developed a staining method using the Stains-all dye, which stains only phosphorylated and sialylated proteins blue and others faint pink (adopted from Sharma et al., 2021). In this way, we were able to distinguish between GMP and, for example, B-LG, which migrates at a similar position in a polyacrylamide gel. This method allows characterisation by size and content of phosphate or sialic acid. Most whey proteins are stained pink, while OPN and GMP are blue. Both staining methods allow subsequent staining with silver nitrate for increased sensitivity.

### Mass Spectrometry

Selected isolates were analysed with mass spectrometry (LC-MS/MS). These data confirmed the presence of osteopontin and kappa-casein, of which GMP is a part.

### Biological Activity of OPN

HEK293 cells were grown in BSA-coated cell culture plates in the presence or absence of the same concentration of a commercial bovine milk OPN (Bio-techne, UK) or our diafiltered OPN isolates from different whey samples. All isolates exhibited cell-adhesive activity.

### General Methods

The invention can be used to fractionate various types of whey or differently pretreated fractions of whey and obtain pure osteopontin and/or glycomacropeptide isolates. Below are presented 35 experiments of isolation of the two proteins at the laboratory scale using 1 mL CIMmultus™ columns.

CIMmultus™ columns are also available as larger industrial scale models, allowing easy scale-up from the laboratory to the industrial scale. By using larger variants of the columns, the invention can be used to produce OPN and GMP at the industrial level as a complementary process in other processes (such as the isolation of lactoferrin, WO2020094731A1).

The process can thus be used in the development of nutritional supplements, special dietary products and drugs.

We performed the following experiments at the laboratory scale:

### Example 1-3

Partially protein-depleted microfiltered acid whey from the dairy, from which lactoferrin was previously removed, was fractionated using strong anion-exchange chromatography using the 1 mL CIMmultus™ QA-1 column. Before loading, we equilibrated the column in buffer A (100 mM sodium citrate pH 5.0) and then loaded 5, 25 or 50 mL of whey. After loading, we washed the column with buffer A. We then eluted the bound proteins with either a linear gradient from 0% to 100% of buffer B (100 mM sodium citrate pH 5.0 with 1 M NaCl) in buffer A or with stepwise pulse elutions with increasing concentration of buffer B. Citrate absorbs in the UV range and is, therefore, less suitable if we want to see the peaks during elution. We analysed the collected fractions by SDS-PAGE and staining with Coomassie brilliant blue R-250 and silver nitrate.

### Example 4

The same whey as used in examples 1-3 (50 mL) was fractionated using weak anion-exchange chromatography using the 1 mL CIMmultus™ DEAE-1 column. The proteins were eluted by stepwise pulse elutions with an increasing concentration of buffer B. We analysed the collected fractions by SDS-PAGE and staining with Coomassie brilliant blue R-250 and silver nitrate.

### Examples 5 and 6

Partially protein-depleted microfiltered acid whey from the dairy, from which lactoferrin had been previously removed, was fractionated using strong anion-exchange chromatography using the 1 mL CIMmultus™ QA-1 column. Before loading, we equilibrated the column in buffer A (50 mM sodium acetate pH 5.0) and then loaded it with 50 or 130 mL of whey. After loading, the column was washed with buffer A. Then the bound proteins were eluted with a linear gradient from 0% to 100% of buffer B (50 mM sodium acetate pH 5.0 with 2 M NaCl) in buffer A. The collected fractions were analysed by RP-HPLC and SDS-PAGE, followed by staining with Stains-all and silver nitrate.

### Examples 7-11

Partially protein-depleted microfiltered sweet whey from the dairy, from which lactoferrin had been previously removed, was fractionated using strong anion-exchange chromatography using the 1 mL CIMmultus™ QA-1 column. Before loading, the column was equilibrated in buffer A (50 mM sodium acetate pH 5.0) and then loaded with 10, 30, 50 or 130 mL of whey. After loading, the column was washed with buffer A. Then the bound proteins were eluted with a linear gradient from 0% to 100% of buffer B (50 mM sodium acetate pH 5.0 with 2 M NaCl) in buffer A. The collected fractions were analysed by RP-HPLC and SDS-PAGE, followed by staining with Stains-all and silver nitrate.

### Example 12

Sweet whey was prepared in laboratory conditions by adding chymosin to milk and microfiltered. The whey was fractionated using strong anion-exchange chromatography using the 1 mL CIMmultus™ QA-1 column. Before loading, the column was equilibrated in buffer A (50 mM sodium acetate pH 5.0) and then loaded with 50 mL of whey. After loading, the column was washed with buffer A. The bound proteins were then eluted with a linear gradient from 0% to 100% of buffer B (50 mM sodium acetate pH 5.0 with 2 M NaCl) in buffer A. The collected fractions were analysed by RP-HPLC and SDS-PAGE and stained with Stains-all and silver nitrate. The purity of the obtained OPN was estimated to be >90% (RP-HPLC) and the purity of GMP to be ˜30% (RP-HPLC).

### Example 13

The same microfiltered sweet whey as in the previous experiment derived from chymosin-treated milk was fractionated using strong anion-exchange chromatography using the 1 mL CIMmultus™ QA-1 column. Before loading, the column was equilibrated in buffer A (100 mM sodium citrate pH 5.0). 37.5 mL of whey was loaded, and the column was washed with buffer A. The bound proteins were eluted with a pulse elution with buffer B (100 mM sodium citrate pH 3.0 with 300 mM NaCl). The collected fractions were analysed by RP-HPLC and SDS-PAGE followed by staining with Stains-all and silver nitrate.

### Example 14-24

In order to determine the binding capacity of the column for OPN, the whey was concentrated: the partially protein-depleted microfiltered acid whey from the dairy, from which lactoferrin had been previously removed, was ultrafiltered with a 10 kDa cut-off membrane to 1/10 or 1/17 of its original volume. This concentrate was then fractionated with strong anion-exchange chromatography using the 1 mL CIMmultus™ QA-1 column. Before loading, the column was equilibrated in buffer A (25 mM sodium acetate pH 5.0). Then 14 mL, 50 mL, 100 ml, 143 mL or 180 mL of the concentrate was loaded, and the flow-through fraction was collected. After loading, the column was washed with buffer A, and the bound proteins were eluted first with a pulse elution with buffer B (25 mM sodium acetate pH 5.0+100 mM NaCl) to remove the weakly bound impurities and unbound substances, then with buffer C (25 mM sodium acetate pH 5.0+400 mM NaCl) to elute OPN and finally with buffer D (25 mM sodium acetate pH 5.0+2M NaCl) to elute the strongly bound impurities. After re-equilibration, all collected flow-through fractions were reloaded, and the elution steps were repeated. This was to check whether any OPN was leftover in the flow-through fractions and whether it could be harvested by re-chromatography. All the eluted fractions were analysed with RP-HPLC and SDS-PAGE and stained with Stains-all and silver nitrate. Around 10-15% of OPN did not bind to the column during the first load but did bind in the second loading round. The purity of OPN was determined to be 57%-88% (RP-HPLC). The highest purity was achieved when loading 100 mL of the concentrated whey. When the amount of whey loaded to the column was increased, it was observed that OPN displaced B-LG and A-LA from the column-the elution fractions containing OPN contained less of these two proteins with increasing the load. The binding capacity of the column for OPN was determined to be >17.5 mg/mL (achieved when loaded with 180 mL of 10× concentrated whey). According to the graph of eluted mass of OPN as a function of the loaded concentrate, the maximum binding capacity has not yet been reached. The identity of OPN was confirmed in the fraction with the highest OPN concentration in the loading of 100 mL concentrate by mass spectrometry.

Fractions containing the most of OPN from all runs were pooled and diafiltered with water using an Amicon spin column with a 10 kDa cut-off membrane. The purity of the diafiltered OPN was 96.9% as judged from RP-HPLC. The diafiltered OPN exhibited cell adhesion activity in the assay with HEK293 cells.

### Example 25-30

In experiments similar to examples 14-24, microfiltered sweet whey, from which lactoferrin had been previously removed, was concentrated and fractionated. The sweet whey was concentrated at 10% of its original volume. This concentrate was fractionated with strong anion-exchange chromatography using the 1 mL CIMmultus™ QA-1 column. Before loading, the column was equilibrated in buffer A (25 mM sodium acetate pH 5.0). 14 mL, 50 mL and 100 mL of the concentrate were loaded, and the flow-through fractions were collected. After loading, the column was washed with buffer A, and the bound proteins were eluted first with a pulse elution with buffer B (25 mM sodium acetate pH 5.0+100 mM NaCl), then with buffer C (25 mM sodium acetate pH 5.0+400 mM NaCl) and finally with buffer D (25 mM sodium acetate pH 5.0+2M NaCl). After re-equilibration, all of the collected flow-through fractions were reloaded, and the elution steps were repeated. All the eluted fractions were analysed with RP-HPLC and SDS-PAGE, followed by staining with Stains-all and silver nitrate. The purity of OPN was determined to be 52%-82%, and GMP's purity was 54%-78% (RP-HPLC). The binding capacity of the column for OPN and GMP was also determined. The highest amount of GMP (9.8 mg/mL column) was isolated when loaded with 50 mL of sweet whey concentrate, and the purest GMP (78%) was isolated when loaded with 14 mL of concentrate. Presumably, with increasing the load, the weaker binding GMP is displaced from the column by stronger binding OPN. The highest amount of OPN (7.2 mg/ml of the column with a purity of 81%) was isolated when loading with 100 ml of the sweet whey concentrate. After this loading, no OPN passed into the flow-through. This means that the low pH of the acid whey was probably responsible for the passage of some OPN into the flow-through fraction in the previous examples. From loading 50 mL of whey, 3.5 mg of OPN was isolated with a purity of 82%.

Fractions containing the most of OPN from all runs were pooled and diafiltered with water using an Amicon spin column with a 10 kDa cut-off membrane. The purity of the diafiltered OPN was 94.1% as judged from RP-HPLC. The diafiltered OPN exhibited cell adhesion activity in the assay with HEK293 cells.

### Example 31

Acid whey was prepared under laboratory conditions by adding lactic acid to skim milk until the pH reached 4.6. We removed the precipitated caseins with a cheesecloth. Lactoferrin was removed from this whey by cation-exchange chromatography, and the resulting flow-through was fractionated with strong anion-exchange chromatography using the 1 mL CIMmultus™ QA-1 column. Before loading, the column was equilibrated in buffer A (25 mM sodium acetate pH 5.0). 120 mL of whey was loaded, and the flow-through fraction was collected. After loading, the column was washed with buffer A and eluted the bound proteins first with a pulse elution with buffer B (25 mM sodium acetate pH 5.0+100 mM NaCl), then with buffer C (25 mM sodium acetate pH 5.0+400 mM NaCl) and finally with buffer D (25 mM sodium acetate pH 5.0+2M NaCl). After re-equilibration, all collected flow-through fractions were reloaded, and the elution steps were repeated. All eluted fractions were analysed with RP-HPLC and SDS-PAGE, followed by staining with Stains-all and silver nitrate. The purity of OPN was determined to be 31%-79% (RP-HPLC). The binding capacity of the column for OPN for this type of whey was also determined. 17.1 mg OPN was isolated from 120 mL of laboratory-made acid whey. During this run, 14% of OPN passed into the flow-through and was isolated in the subsequent fractionation of the flow-through. The concentration of OPN (as inferred from RP-HPLC by comparing the isolate to the full-length commercial bovine OPN of known concentration) in this laboratory-made whey was comparable to the concentration of OPN in the 17-fold concentrated acid whey from the dairy, and was therefore presumably 17-fold higher than in the original whey from the dairy (assuming no losses occurred during the concentration step). The concentration of OPN in acid whey prepared was 9-fold higher than that reported in the literature for milk (Schack et al., 2009) and was in the same range as lactoferrin concentrations (Cheng et al., 2008). The identity of OPN in the fraction with the highest OPN concentration was confirmed with mass spectrometry. The fraction containing the most of OPN was diafiltered with water using an Amicon spin column with a 10 kDa cut-off membrane. The purity of the diafiltered OPN was 96.9% as judged from RP-HPLC. The diafiltered OPN exhibited cell adhesion activity in the assay with HEK293 cells.

### Examples 32-35

Different types of whey were prepared on a laboratory scale. First, skim milk was prepared by two cycles of centrifugation of raw milk and removing the fat at the end of each cycle. Lactic acid was then added to the skim milk to prepare acid whey (whey 1 with pH 4.7) or chymosin to prepare sweet whey (whey 3 with pH 6.7). To a portion of sweet whey, lactic acid was added to a pH of 4.7 (whey 4) or pH 5.34 (whey 5). The precipitated caseins were removed by filtering through cheesecloth.

150 mL of each whey was fractionated as in examples 14-31. Before loading, the column was equilibrated in buffer A (25 mM sodium acetate pH 5.0). 120 mL of whey was loaded, and the flow-through fraction was collected. After loading, the column was washed with buffer A and eluted the bound proteins first with a pulse elution with buffer B (25 mM sodium acetate pH 5.0+100 mM NaCl), then with buffer C (25 mM sodium acetate pH 5.0+400 mM NaCl) and finally with buffer D (25 mM sodium acetate pH 5.0+2M NaCl). After re-equilibration, all collected flow-through fractions were reloaded, and the elution steps were repeated. All the eluted fractions were analysed with RP-HPLC and SDS-PAGE, followed by staining with Stains-all and silver nitrate. 0.5 mg of OPN was obtained from whey 1. 4.3 mg of OPN and 2.3 mg of GMP were obtained from whey 3. 13.4 mg of OPN was obtained from whey 4. 15.4 mg of OPN was obtained from whey 5. The purity of OPN ranged from 34.6% to 58.6% according to RP-HPLC, and was >90% according to SDS-PAGE. The purity of GMP was 58.8% (RP-HPLC). The laboratory-produced whey again contained 10 times more OPN than the whey from the dairy. The identity of OPN in the fraction with the highest OPN content in whey 4 was confirmed by mass spectrometry.

The fractions containing the most of OPN were diafiltered with water using an Amicon spin column with a 10 kDa cut-off membrane. The purity of the diafiltered OPN from whey 1, whey 3, whey 4 and whey 5 was 97.6%, 94.2%, 98.4% and 95.5%, respectively, as judged from RP-HPLC. The diafiltered OPN exhibited cell adhesion activity in the assay with HEK293 cells.

### Examples 36-49

Different types of whey were prepared from the same batch of milk from the dairy on a laboratory scale. Part of the milk was pasteurised and skimmed by the dairy, and part was unpasteurised, and skimmed in our laboratory as before. We prepared and microfiltered (0.2 μm) the following whey samples:
Chymosin-derived whey from pasteurised milk: pH adjusted to 5.0
Acid whey from unpasteurised milk: addition of lactic acid until pH 4.7, then raising pH to 5.3 with NaOH
Acid whey from pasteurised milk: addition of lactic acid until pH 4.7
Acid whey from pasteurised milk: addition of lactic acid until pH 4.7, then raising pH to 5.0 with NaOH
Acid whey from pasteurised milk: addition of lactic acid until pH 4.7, then raising pH to 5.3 with NaOH.

We then performed 2 fractionation experiments on 100 mL of whey with the 1-mL CIMmultus™ QA-1 column for each set of conditions (7 conditions in total) in a total of 14 experiments. The experiments were performed as before, with minor differences. We tested different pulse elution conditions (elution with low pH only, elution with salt without buffer, and elution with buffer and salt). All eluted fractions were analysed by RP-HPLC, and the mass and purity of the isolated OPN were determined. We obtained up to 5.8 mg of OPN with a purity of up to 79.5%. Washing the column with low pH buffer prior to elution of OPN increased the purity of OPN. Saline water alone (0.425 M NaCl) eluted OPN with the same efficiency as a buffer with salt, which may reduce production costs. The pH of whey affected the OPN yield: at pH 4.7 and 5.0, OPN yield was 38% and 32% lower, respectively, than at pH 5.3. We obtained 40% less OPN (with 23% lower purity) from chymosin whey than from acid whey at the same pH. More peptides, IgG and LPO bound to the column in chymosin whey than in acid whey. We isolated 27% more OPN (with 10% higher purity) from unpasteurised milk than from pasteurised milk, suggesting that OPN could be partially removed from the whey fraction by heat treatment. Based on these results, non-heat-treated acid whey with a pH 5.3 is the best starting material.

### Examples 50-53

We scaled up the process using the 80-mL CIMmultus™ QA-80 column, a Cole-Parmer peristaltic pump, a pressure gauge, and a Knauer UV detector. We prepared acid whey (44 L) with a pH of 4.7 from 50 L of pasteurised skimmed milk from the dairy. After removal of the curds, we raised the pH to 5.3 with NaOH. We filtered the whey using a gradient ceramic membrane with 0.5 μm pores and produced 42 L of permeate. In 4 experiments, we fractionated 8.4-9.6 L (a total of 37.2 L) of filtered whey. We optimised the NaCl concentration in the elution and regeneration buffers. We analysed the fractions with RP-HPLC and SDS-PAGE. We obtained 324.0 mg-420.9 mg OPN with purity of 70.7%-86.6% from each run. We pooled the elutions (total mass of OPN 1.47 g (RP-HPLC)) and diafiltered them to a conductivity of 0.355 mS/cm using a Watersep Hollow fibre membrane (10 kDa cut-off) and demineralised water. We freeze-dried the diafiltration retentate and analysed the obtained OPN powder using SDS-PAGE and RP-HPLC. We obtained 1.94 g of the dried protein sample, which contained 83% OPN. SDS-PAGE showed the same three bands as before.

### Reference

Azuma et al. (2006) “A rapid method for purifying osteopontin from bovine milk and interaction between osteopontin and other milk proteins”. International Dairy Journal 16, 370-378.
Bayless et al. (1997) “Isolation and biological properties of osteopontin from bovine milk”. Protein expression and purification 9, 309-314.
Bissonnette et al. (2012) “Proteomic analysis and immunodetection of the bovine milk osteopontin isoforms”. Journal of Dairy Science 95, 567-579.
Bobe et al. (1998) “Separation and Quantification of Bovine Milk Proteins by Reversed-Phase High-Performance Liquid Chromatography”. Journal of agricultural and food chemistry 46, 458-463.
Cheng et al. (2008) “Factors affecting the lactoferrin concentration in bovine milk”. Journal of dairy science 91, 970-976.
Christensen et al. (2020) “Milk osteopontin retains integrin-binding activity after in vitro gastrointestinal transit”. Journal of dairy science 103, 42-51.
Laemmli (1970) “Cleavage of Structural Proteins during the Assembly of the Head of Bacteriophage T4”. Nature 227, 680-685.
Schack et al. (2009) “Considerable variation in the concentration of osteopontin in human milk, bovine milk, and infant formulas”. Journal of dairy science 92, 5378-5385.
Sharma et al. (2021) “Distinction between glycomacropeptide and β-lactoglobulin with ‘stains all’ dye on tricine SDS-PAGE gels”. Food Chemistry 340, 127923.

1. A process for the isolation of osteopontin (OPN) or glycomacropeptide (GMP) from whey or a whey-derived liquid comprising the steps of absorption onto a monolithic chromatography column, an optional washing of the column and subsequent elution of said OPN or GMP using a change in salt concentration and/or pH.

2. The process according to claim 1, wherein the monolithic column is loaded with whey or whey-derived liquid with such flow rate and in such quantity that OPN displaces more weakly binding substances from the column.

3. The process according to claim 1, wherein the monolithic column is loaded with whey or whey-derived liquid with a flow rate in the range from about 0.1 mL per mL of column volume per minute to about 20 mL per mL of column volume per minute, or in the range from about 0.1 mL per mL of column volume per minute to about 5 mL per mL of column volume per minute.

4. The process according to claim 1, wherein said elution comprises the use of an eluent having a higher ionic strength than said whey or whey derived liquid loaded onto said monolithic chromatography column.

5. The process according to claim 1, wherein said elution comprises the use of an eluent having a pH lower than the pH of said whey or whey-derived liquid loaded onto said monolithic chromatography column.

6. The process according to claim 1, wherein said elution is performed by the use of a change in salt concentration and a change in pH.

7. The process according to claim 6, wherein the elution is performed by a step change of the salt concentration and a gradient change of the pH.

8. The process according to claim 6, wherein the elution is performed by a step change of the pH and a gradient change of the salt concentration.

9. The process according to claim 1, wherein OPN and GMP are eluted separately such that separate fractions are obtained where one fraction comprises the majority of said OPN and a second fraction comprises the majority of said GMP.

10. The process according to claim 1, wherein said absorption onto a monolithic chromatography column is a high-performance liquid chromatography (HPLC), such as anion-exchange HPLC.

11. The process according to claim 1, wherein said whey or whey derived liquid is filtered prior to said absorption, such as by microfiltration.

12. The process according to of claim 1, wherein said monolithic chromatography column following the elution is regenerated by a solution having a concentration of sodium chloride in the range from about 0.8M to about 2.0M, and optionally pH in the range from about 2.5 to about 3.5.

13. A method for preparing OPN or GMP comprising the process as defined in claim 1.

14. The method according to claim 13, further comprising a desalination step.

15. The method according to claim 13, comprising a further chromatography step.

16. The method according to claim 13, further comprising a lyophilisation, spray drying or other form of drying to obtain a solid OPN or GMP product.
